(19) United States
(12) Patent Application Publication
(10) Pub. No.: US 2007/0092904 A1
(43) Pub. Date: Apr. 26, 2007

(54) Method for preparing limiting quantities of nucleic acids

(75) Inventor: Jeffrey R. Shearstone, Cambridge, MA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec MA Inc., Cambridge, MA (US)

(21) Appl. No.: 11/581,077

(22) Filed: Oct. 16, 2006

Related U.S. Application Data
(60) Provisional application No. 60/727,868, filed on Oct. 19, 2005.

(51) Int. Cl. C12Q 1/68; C12P 19/34

(52) U.S. Cl. 435/6; 435/91.2

(57) ABSTRACT

The present invention relates generally to the amplification of nucleic acids. More specifically, the present invention facilitates amplification of total RNA for a variety of purposes, including analysis utilizing nucleotide assays, constructing cDNA libraries, in situ hybridization, and TaqMan. Additionally, the present invention facilitates amplification of total RNA isolated from biological tissues.

### CROSS-REFERENCE TO RELATED APPLICATION

This application claims the benefit of U.S. Provisional Patent Application No. 60/727,868, filed Oct. 19, 2005, herein incorporated by reference.

### BACKGROUND OF THE INVENTION

1. Field of the Invention

The present invention relates generally to the amplification of nucleic acids. More specifically, the present invention facilitates amplification of total RNA for a variety of purposes, including analysis utilizing nucleotide assays, constructing cDNA libraries, in situ hybridization, and TaqMan. Additionally, the present invention facilitates amplification of total RNA isolated from biological tissues.

2. Background Art

Many biological functions are accomplished by altering the expression of various genes through transcriptional (e.g. through control of initiation, provision of RNA precursors, RNA processing, etc.) and/or translational control. For example, fundamental biological processes such as cell cycle progression, cell differentiation and cell death, are often characterized by the variations in the expression levels of a group of genes.

Highly parallel methods of monitoring the expression of a large number of genes in a biological sample are a valuable research and diagnostics tool. However, the amount of starting material that can be obtained from a given source is often limited and it is useful to amplify genetic material prior to analysis. Methods of amplifying the genetic material that allow analysis of a sample that may be too small for analysis without amplification facilitate the analysis of gene expression in small samples and possibly in a single cell.

One method utilized to amplify genetic material is described in Schlingemann et al., “Effective Transcriptome Amplification for Expression Profiling on Sense-Oriented Oligonucleotide Microarrays,” Nuc. Acids Res., 33:e29 (February 2005). Schlingemann et al. utilized a method in which cDNA was generated from isolated mRNA. Id., FIG. 1. This generated cDNA was then transcribed into antisense RNA. Id. Sense cDNA was then generated from the antisense RNA. Id. Schlingemann et al. then generated fluorescent dye-labeled antisense cDNA from sense cDNA utilizing Klenow fragment of E. coli DNA polymerase I. Id. Schlingemann et al., determined that this method was able to amplify and label as little as 2 ng of total RNA. Id., p. 11.

U.S. Pat. Nos. 5,545,522; 5,716,785; 5,891,636; and 6,291,170B1 describe a process for amplifying nucleic acids comprising synthesizing a nucleic acid by hybridizing an oligonucleotide primer complex to the sequence and extending the oligonucleotide primer to form a first strand complementary to the sequence, as well as synthesizing a second strand complementary to the first strand. The oligonucleotide primer complex comprises an oligonucleotide primer complementary to the sequence and a promoter region in anti-sense orientation with respect to the sequence. Then, copies of an antisense RNA are transcribed off of the second strand. The transcription step is the step in which a label may be incorporated.

### BRIEF SUMMARY OF THE INVENTION

In one embodiment, the invention is directed to a method for amplifying a nucleic acid population comprising: (a) generating a DNA population from said nucleic acid population; (b) generating an antisense cRNA population from said DNA population; (c) generating a sense cDNA population from said antisense cRNA population; and (d) generating an antisense cDNA population from said sense cDNA population.

In another embodiment, the invention is directed to a method for amplifying an RNA population comprising: (a) generating a cDNA population from said population of RNA; (b) generating an antisense cRNA population from said cDNA population; (c) generating a sense cDNA population from said antisense cRNA population; (d) generating a second antisense cRNA population from said sense cDNA population; (e) generating a second sense cDNA population from the second antisense cRNA population; and (f) generating an antisense cDNA population from the second sense cDNA population.

In a third embodiment, the invention is directed to a method for amplifying a total RNA sample comprising: (a) contacting an RNA population comprising a plurality of different RNAs with a first oligonucleotide primer complex comprising an oligonucleotide primer and an RNA polymerase promoter; (b) extending the oligonucleotide primer in a reaction mixture comprising reverse transcriptase to yield RNA:cDNA duplexes; (c) synthesizing second strand cDNA by incubating the RNA:cDNA with a reaction mixture comprising DNA polymerase to yield cDNA; (d) contacting the cDNA with random oligonucleotide primers; and (e) generating antisense cDNA from the cDNA by extending the random oligonucleotide primers in a reaction mixture comprising a molecule with polymerase activity. Optimally, the following additional steps may be incorporated in the method between steps (c) and (d) above: (i) producing an antisense cRNA by incubating the cDNA in a reaction mixture comprising an RNA polymerase; (ii) contacting the antisense cRNA with a reaction mixture comprising random oligonucleotide primers; (iii) generating RNA:cDNA duplexes from the antisense cRNA by extending the random oligonucleotide primers in a reaction mixture comprising a reverse transcriptase; (iv) contacting the cDNA with a second oligonucleotide primer complex comprising an oligonucleotide primer and an RNA polymerase promoter and extending the oligonucleotide primer to generate a second cDNA; (v) producing a second antisense cRNA by an in vitro transcription reaction; (vi) contacting the second antisense cRNA with random oligonucleotide primers; and (vii) generating RNA:cDNA duplexes from the second antisense cRNA by extending the random oligonucleotide primers in a reaction mixture comprising a reverse transcriptase.

In a fourth embodiment, the invention is directed to a method for evaluating the nucleic acid in a sample comprising: (a) contacting an RNA population comprising a plurality of different RNAs with a first oligonucleotide primer complex comprising an oligonucleotide primer and an RNA polymerase promoter; (b) extending the oligonucleotide primer in a reaction mixture comprising reverse transcriptase to yield RNA:cDNA duplexes; (c) synthesizing second strand cDNA by incubating the RNA:cDNA with a reaction mixture comprising DNA polymerase to yield cDNA; (d) contacting the cDNA with random oligonucleotide primers; (e) generating antisense cDNA from the cDNA by extending the random oligonucleotide primers in a reaction mixture comprising a molecule with polymerase activity; and (f) utilizing a nucleotide assay. Optimally, the following additional steps may be incorporated between steps (c) and (d) of the method above: (i) producing an antisense cRNA by incubating the cDNA in a reaction mixture comprising an RNA polymerase; (ii) contacting the antisense cRNA with a reaction mixture comprising random oligonucleotide primers; (iii) generating RNA:cDNA duplexes from the antisense cRNA by extending the random oligonucleotide primers in a reaction mixture comprising a reverse transcriptase; (iv) contacting the cDNA with a second oligonucleotide primer complex comprising an oligonucleotide primer and an RNA polymerase promoter and extending the oligonucleotide primer to generate a second cDNA; (v) producing a second antisense cRNA by an in vitro transcription reaction; (vi) contacting the second antisense cRNA with random oligonucleotide primers; (vii) generating RNA:cDNA duplexes from the second antisense cRNA by extending the random oligonucleotide primers in a reaction mixture comprising a reverse transcriptase.

### BRIEF DESCRIPTION OF THE FIGURES

FIG. 1 shows a paradigm for production of amplified, anti-sense cDNA in which cDNA and cRNA are each only generated once before generation of the sense cDNA.

FIGS. 2(A and B) shows a paradigm for production of amplified, anti-sense cDNA in which cDNA and cRNA are each generated twice before generation of the sense cDNA.

FIGS. 3(A and B) shows a comparison between the method of the present invention and other methods currently utilized in the field. For all panels, the dotted line represents average replicate values from 1 μg total RNA using One-Cycle Target Labeling as described in the Affymetrix Eukaryotic Sample Analysis Technical Manual, Rev 5 (“AFFX 1rd”). Data for the amplification method of the present invention, as described in FIG. 2 (“BIIB 3rd”) (diamond), Two-Cycle Target Labeling as described in the Affymetrix Eukaryotic Sample Analysis Technical Manual, Rev 5 (“AFFX 2rd”) (square), Arcturus RiboAmp HS (“ARC HS”) (circle), and NUGEN Ovation (“NGN”) (triangle) is presented as average value of all replicates. Error bars are included for the data from the amplification method of the present invention where n=4, otherwise n=2. (A) Number of Affymetrix GeneChips that can be hybridized from amplified product as a function of total RNA starting mass. The y-axis has been normalized because the amplification methods that generate a cDNA target require only 2 μg nucleic acid for hybridization, but 11 μg of cRNA target is required for an equivalent performance. (B) Percent of probe sets called present as a function of total RNA starting mass. (C) Target degradation plots for 5 ng of total RNA using the AffyRNAdeg function of the Bioconductor Affy module. Gentleman et al., “Bioconductor: Open Software Development for Computational Biology and Bioinformatics,” Genome Biol., 5:R80 (2004); Gautieret al., “Affy—Analysis of Affymetrix GeneChip Data at the Probe Level,” Bioinformatics, 20:307-15 (2004). The perfect match intensity for individual probes in a probe set are ordered by location relative to the 5′ end of the targeted RNA molecule. The average intensity for all probe locations is presented for each amplification protocol. The slope of each line is directly linked to the severity of truncation and thus inversely proportional to the average target length. (D) Accuracy of exogenous RNA spikes for 5 ng of total RNA sample. 15 fg, 4 fg, 2 fg, and 1 fg of B. subtilis transcripts dap, thr, phe, and lys, respectively, were added to 5 ng of total RNA. A linear regression of expression intensity versus mass of the spike would ideally produce a slope and R² value of one. The amplification method of the present invention shows equivalent or superior performance in all benchmarking criteria compared to the other amplification protocols.

FIG. 4 shows the inter-sample precision. The average log intensity was calculated for replicates (n=4) at each starting mass of total RNA. Probe set rows were then ordered by descending average intensity of all samples and false colored based on expression values for each individual probe set. White, gray, and black coloring represent high (log₂ RFU=16), mid (log₂ RFU=12), and low (log₂ RFU=8) gene expression intensity, respectively. Inter-sample precision is roughly maintained across all starting masses, although the number of high and mid expressing genes is slightly reduced at 50 pg and substantially reduced when starting with 10 pg of total RNA.

FIG. 5 shows a titration of the amount of starting material utilized in the present invention. Four replicate samples of starting from 5 ng, 1 ng, 0.5 ng, 100 pg, 50 pg, or 10 pg of mouse universal total RNA was processed using the amplification method of the present invention. (A) Heat map of expression intensity for all probe sets and samples. Probe set rows were ordered by descending average intensity of all samples then false colored based on expression values for each individual probe set. White, gray, and black coloring represent high (log₂ RFU=16), mid (log₂ RFU=12), and low (log₂ RFU=8) gene expression intensity, respectively. Averaging the replicates reveals consistent expression intensity as low as 50 pg of starting total RNA. Low expressing probe sets at 5 ng, 1 ng, 0.5 ng, and 100 pg were the first to be dropped from expression profiles at 50 pg and 10 pg of starting total RNA.

FIGS. 6(A and B) shows the intra-sample and inter-sample precisions of the method of the present invention. (A) Intra-sample precision. The coefficient of variation and average expression intensity for each probe set was calculated across replicate hybridizations (n=4) starting from 5 ng, 1 ng, 0.5 ng, 100 pg, 50 pg, or 10 pg mouse universal total RNA (black traces, lower left to upper right, respectively). The same analysis was conducted using four replicate hybridizations from 1 μg of mouse universal total RNA using AFFX 1rd (gray trace). The amplification method of the present invention, as described in FIG. 2, performed similarly to AFFX 1rd from as little as 500 pg of sample. (B) Stochastic effects of dilution. The coefficient of variation for each exogenous spike present between 1000 and 8 copies, for starting total RNA mass at and below 500 pg, was plotted as a function of copy number (triangles). The theoretical deviation due to the stochastic variance inherent in working with highly diluted samples of limiting material can be estimated by a Poisson distribution (squares). Stenman, J. and Orpana, A., “Accuracy in Amplification,” Nat Biotechnol., 19:1011-12 (2001). The slope of the linear regression through the experimental data is virtually identical to that of the Poisson distribution data, indicating that the reduction in intra-sample precision seen below 500 pg of total RNA is entirely due to dilution effects, rather than a limitation of the amplification method of the present invention. (C) Inter-sample precision. Average adjusted expression intensities were plotted as a function of starting mass of total RNA. Eight probe sets representing housekeeping genes at various relative expression intensities were chosen for visualization: GAPD (diamond), RPL13A (triangle), ACTB (X), HPRT1 (dash), YWHAZ (plus), TFRC (square), HMBS (*), and PCX (circle). A linear regression yields slope and R² values close to the ideal value of 1. Similarly, calculation of slope and R² for the highest expressing 11,300 probe sets yielded average values of 1.02±0.11 and 0.99±0.01, respectively, indicating virtually no amplification bias when working within a range of 5 ng to 50 pg of starting total RNA.

FIG. 7 shows the accuracy and linear limits of detection using exogenous control transcripts. Four replicate samples of 5 ng, 1 ng, 0.5 ng, 100 pg, or 50 pg of total RNA was processed using the amplification method of the present invention, as indicated in FIG. 2. Adjusted intensities were converted to log values and replicates were averaged. A mass titration of dap, thr, phe, and lys transcripts were spiked at equal molar ratios relative to four replicates samples containing 5 ng (squares), 1 ng (diamond), 0.5 ng (triangle), 100 pg (circle), or 50 pg (cross) mouse universal total RNA. A linear regression of adjusted intensity versus absolute mass of spiked transcript produces a slope of 1.02 and R²=0.99. The arrow denotes the linear limit of detection at 0.02 fg, corresponding to an absolute copy number of 20.

FIG. 8 shows the accuracy of the method of the present invention by QRT-PCR validation using a total RNA tissue panel. QRT-PCR primer sets were designed using the Affymetrix probe set consensus sequence of eight housekeeping genes: GAPD (diamond), RPL13A (triangle), ACTB (cross), HPRT1 (dash), YWHAZ (plus), TFRC (square), HMBS (asterisk), and PCX (circle). 1 μg or 500 pg total RNA from mouse brain, embryo, heart, kidney, liver, lung, ovary, and spleen was processed using AFFX 1rd or the amplification method of the present invention, as indicated in FIG. 2 (“BIIB 3rd”), respectively. For each probe set, 28 pairwise tissue expression ratios were created. Results from the AFFX 1rd (A) and the amplification method of the present invention, as described in FIG. 2 (“BIIB 3rd”) (B) were plotted against values obtained by QRT-PCR.

FIG. 9 shows the B-cell and T-cell enriched genes that were identified using the method of the present invention, as described in FIG. 2, on 100 primary cells flow sorted from mouse spleen. 100 total cells (n=2) or 500 total cells per sample (n=1) was sorted directly into lysis buffer. Using TCR and B220 marker staining, each sample well received a titration of T or B cells (columns), which was subsequently purified and processed by the method of the present invention, as described in FIG. 2. The heat map represents gene expression values for a subset of 51 T-cell or 64 B cell specific qualifiers identified by quantitative trait analysis. False coloring is on a global basis with white, gray, and black coloring represents relatively high, mid, and low expression, respectively. Results are consistent across the 100 cell and 500 cell titration. This list contains the known T-cell markers CD3, CD8, CD27, and the T-cell receptor alpha and beta chains (top panel), as well as the known B-cell markers CD22, CD24, several Histocompatibility 2 class II family members, and many Immunoglobulin family members (bottom panel).

FIG. 10: shows the B-cell and T-cell enriched genes identified using the method of the present invention, as described in FIG. 2, on a titration of 100 or 500 primary flow sorted cells. 100 total cells (n=2) or 500 total cells per sample (n=1) was sorted directly into lysis buffer. Using TCR and B220 marker staining, each sample well received a titration of T or B cells (columns), which was subsequently purified and processed by the method of the present invention, as described in FIG. 2. Using the 100 cell titration data, a quantitative trait analysis yielded a list that contained 25 T-cell specific and 31 B-cell specific transcripts, after duplicate qualifiers were removed. Using the 500 cell titration data, the same analysis generated a list that contained 32 T-cell and 41 B-cell specific qualifiers. The union of these two lists yielded 51 T-cell and 64 B-cell qualifiers, indicating little overlap between the two lists. However, this expression intensity heat map of the 115 qualifiers illustrates the high degree of concordance between the 100 and 500 cell data, suggesting that the 99% confidence interval and 10 fold change cut-off eliminated biologically relevant qualifiers from both lists. False coloring is on a global basis with white, gray, and black coloring represents relatively high, mid, and low expression, respectively.

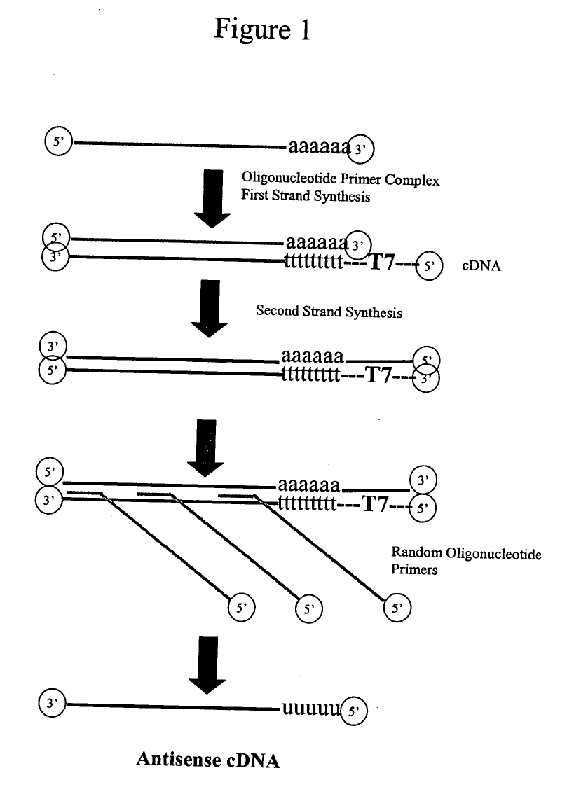

### DETAILED DESCRIPTION OF THE INVENTION

Terminology

As used herein in, the singular form “a,” “an,” and “the” include plural references unless the context clearly dictates otherwise. For example, the term “an agent” includes a plurality of agents, including mixtures thereof.

As used herein, the terms “nucleic acid”, “nucleic acids”, “nucleic acid population”, and “nucleic acid populations” may include any polymer or oligomer of pyrimidine and purine bases, such as cytosine, thymine, and uracil, and adenine and guanine, respectively. The terms “nucleic acid” and “nucleic acids” contemplates any deoxyribonucleotide or ribonucleotide component, and any chemical variants thereof, including, but not limited to methylated, hydroxmethylated or glucosylated forms. The polymers or oligomers may be heterogeneous or homogeneous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. Oligonucleotide and polynucleotide are included in this definition and relate to two or more linked nucleic acids.

As used herein, the term “RNA population” may include total RNA, polyadenylated RNA, non-polyadenylated RNA, mRNA, or rRNA The RNA population may be heterogeneous, referring to any mixture of two or more distinct species of RNA. The species in the RNA population may be distinct based on any chemical or biological differences, including differences in base composition, length, or conformation. The RNA population may contain full length mRNAs or mRNA fragments (i.e., less than full length) resulting from in vivo, in situ, or in vitro transcriptional events involving corresponding genes, gene fragments, or other DNA templates. In one embodiment, the RNA population of the present invention may contain single-stranded polyadenylated RNA, which may be obtained from an RNA mixture (e.g., a whole cell RNA preparation).

As used here, the terms “cRNA” and “cRNA population” refer to RNA produced from a DNA or cDNA template. In one embodiment, in vitro transcription is utilized to synthesize the cRNA.

As used herein, the term “DNA population” refers to single-stranded or double-stranded DNA. The DNA population may be heterogeneous, referring to any mixture of two or more distinct species of single-stranded DNA or double-stranded DNA, which may include DNA representing genomic DNA, genes, gene fragments, oligonucleotides, PCR products, expressed sequence tags, or nucleotide sequences corresponding to known or suspected single nucleotide polymorphisms (“SNPs”), having nucleotide sequences that may overlap in part or not at all when compared to one another. The species may be distinct based on any chemical or biological differences, including differences in base composition, order, length, or conformation. The single-stranded DNA population may be isolated or produced according to methods known in the art, and may include single-stranded DNA isolated from double-stranded DNA, or single-stranded DNA synthesized as an oligonucleotide. The double-stranded DNA population may also be isolated according to methods known in the art, such as PCR or reverse transcription.

As used herein, the terms “cDNA” and “cDNA population” refer to single-stranded or double-stranded DNA produced from an RNA template.

The cDNA of the present invention may be produced according to any method known in the art. In one embodiment of the invention, an RNA population, such as polyadenylated RNA may be used to produce the corresponding cDNA in the presence of reverse transcriptase, oligonucleotide primers and deoxyribonucleoside triphosphates (“dNTPs”).

As used herein, the terms “oligonucleotide primer”, “oligonucleotide primers”, “random oligonucleotide primer”, and “random oligonucleotide primers” refer to an oligonucleotide containing anywhere from about 6 to about 30 nucleotides in any combination. In one embodiment, the oligonucleotide primer contains about 10 to about 30 nucleotides. In another embodiment, the oligonucleotide primer contains about 18 to about 25 nucleotides. In yet another embodiment, the oligonucleotide primer is an oligonucleotide poly d(T) primer. In still yet another embodiment, the oligonucleotide primer is a hexamer.

As used herein, the terms “oligonucleotide poly d(T) primer” and “oligonucleotide poly d(T) primers” refer to an oligonucleotide containing anywhere from about 10 to about 30 thymidine residues. In another embodiment, the oligonucleotide contains about 18 to about 25 thymidine residues. The oligonucleotide poly d(T) primer may optionally contain adenine, guanidine, or cytosine residues, in addition to the thymidine residues.

As used herein, the terms “hexamer”, “hexamers”, “random oligonucleotide hexamer” and “random oligonucleotide hexamers” refer to an oligonucleotide containing 6 nucleotides in any combination.

As used herein, the term “oligonucleotide primer complex” refers to an oligonucleotide primer linked to a promoter region. In one embodiment, the promoter region is an RNA T7 polymerase promoter region.

As used herein, the term “reverse transcriptase” may be any enzyme that is capable of synthesizing a corresponding cDNA from an RNA molecule in the presence of the appropriate oligonucleotide primers and nucleoside triphosphates (“NTPs”). In one embodiment, the reverse transcriptase may be from avian myeloblastosis virus (“AMV”), Moloney murine leukemia virus (“MMLV”) or Rous Sarcoma Virus (“RSV”). In another embodiment, the reverse transcriptase may be a thermal stable enzyme, including a thermal stable MMLV.

As used herein, the term “complementary to” refers to a sequence that will form specific base pairing that includes e.g., Watson-Crick base pairing, as well as other forms of base pairing such as Hoogsteen base pairing, with a nucleic acid of interest.

As used herein, the term “sense cDNA” refers to a DNA that has the same nucleotide sequence as the initial RNA molecule, but includes thymine in place of uracil.

As used herein, the terms “antisense cDNA” and “second antisense cDNA” refer to a DNA that is complementary to the initial RNA molecule.

As used herein, the terms “antisense RNA”, “antisense cRNA”, “second antisense RNA”, and “second antisense cRNA” refers to an RNA molecule that is complementary to the initial RNA molecule.

As used herein, the term “nucleotide assay” comprises any gene expression monitoring system. The term “nucleotide assay” includes, but is not limited to, a nucleotide array (including, but not limited to, an oligonucleotide array, a cDNA array, and a spotted array), a membrane blot (such as used in hybridization analysis such as Northern, Southern, dot, and the like), a microwell, a sample tube, a bead, a fiber (or any solid support comprising bound nucleic acids), a TaqMan analysis, or an in situ hybridization. The gene expression monitoring system may also comprise nucleic acid probes in solution.

As used herein, the term “nucleotide array” comprises a support, such as a solid support, with polynucleotide probes attached to the support. A nucleotide array will typically comprise a plurality of different polynucleotide probes that are coupled to a surface of the support in different, known locations. Other terms for “nucleotide array” include “microarrays” or “chips”.

As used herein, the term “TaqMan” application refers to a polymerase chain reaction (“PCR”) that utilizes fluorescent probes to measure the amounts of nucleic acids in a sample. D. W. Watson & B. Li., “TaqMan Applications in Genetic and Molecular Toxicology,” Intl. J. Tox., 24:139-145, 139 (May-June 2005). In one embodiment, a fluorogenic probe containing a reporter dye and a quencher dye anneals to the nucleic acid of interest. Id. Then, the 5′-exonuclease activity of the Taq polymerase may cleave the hybridized fluorogenic probe during the extension phase of the PCR cycle. Id. The cleavage reaction yields a dissociated reporter dye from the quencher, resulting in an increase in detectable fluorescence. Id.

In one embodiment of the present invention, the cDNA produced from an RNA population as a template may be isolated from any resulting RNA:DNA heteroduplexes by heat or enzyme treatment (e.g., RNase H). Fragments of the cDNA population may be created by treatment with any enzyme capable of cleaving single-stranded DNA. Examples of such enzymes may include, but are not limited to, some restriction enzymes (e.g., HaeIII) and DNase I. The digestion conditions (e.g., time and enzyme concentration) may be regulated to achieve the desired amount and type of fragmentation. Fragments of the single-stranded cDNA population may also be created by physical methods such as sonication or shearing. In addition, fragments of the single-stranded cDNA population may be produced by fragmentation of double-stranded DNA followed by denaturation to obtain single-stranded DNA.

The present invention provides a method for amplifying nucleic acids. More specifically, the present invention facilitates amplification of total RNA for a variety of purposes, including analysis utilizing nucleotide assays.

Generally, the method of the present invention involves generating a DNA population from a nucleic acid population. Then, an antisense cRNA population may be generated from the DNA population. Next, a sense cDNA population may be generated from the antisense RNA population. Finally, an antisense cDNA population is produced from the sense cDNA population.

In one embodiment, a DNA population is generated from a nucleic acid population. Then, an antisense cRNA population may be generated from the DNA population. Next, a cDNA population may then be generated from the antisense cRNA population. A second antisense cRNA population may then be generated from the cDNA population. Then, a sense cDNA population may be generated from the second antisense cRNA population. Finally, an antisense cDNA population is produced from the sense cDNA population.

The method of the present invention also involves amplifying nucleic acids to utilize in nucleotide assays.

Generally, the method of the present invention involves generating an antisense cDNA population, as described above, and then utilizing that antisense cDNA in a nucleotide assay. In one embodiment, a labeled antisense cDNA population may be generated, as described above, and then utilized to analyze RNA levels in a tissue. In one embodiment, the nucleotide assay comprises a nucleotide array. In another embodiment, the nucleotide assay comprises a bead. In still yet another embodiment, the nucleotide assay comprises a TaqMan analysis.

Generation of a DNA Population from a Nucleic Acid Population

The starting nucleic acid population may be a DNA population. The DNA population may be isolated from, or derived from, a nucleic acid population by any method known in the art. For example, the DNA population may be single-stranded cDNA produced from an mRNA template, single-stranded DNA isolated from double-stranded DNA, or single-stranded DNA synthesized as an oligonucleotide. The DNA population may also be isolated according to methods known in the art, such as PCR and reverse transcription. Alternatively, the DNA population may be derived from genomic DNA, genes, gene fragments expressed sequence tags, or nucleotide sequences corresponding to known or suspected SNPs, having nucleotide sequences that may overlap in part or not at all when compared to one another.

If the starting nucleic acid population is an RNA population, the RNA population may be obtained or derived from any tissue or cell source, including plant, viron, bacteria, fungi, or algae. In one embodiment, the tissue is eukaryotic. In another embodiment, the tissue is human.

The tissue or cell source may include, but is not limited to, a fine needle aspiration, a laser capture microdissection, a tissue sample isolated utilizing laser scanning cytometry, a tissue sample isolated utilizing flow cytometry, a tissue biopsy sample, a cell sorted population, cell culture, or a single cell. In one embodiment, the tissue sample may include, but is not limited to, brain, liver, heart, kidney, lung, spleen, retina, bone, lymph node, endocrine gland, reproductive organ, blood, nerve, vascular tissue, and olfactory epithelium. In another embodiment, the tissue or cell source may be embryonic or tumorigenic.

The RNA population may be obtained from, or derived from, the tissue of interest by any means known in the art. For example, the mRNA may be isolated from the sample of interest using an acid guanidinium-phenyl-chloroform extraction method following by oligo dT column chromatography or use of (dT)ₙ magnetic beads.

The cDNA population may be generated from the RNA population by any means known in the art.

In one embodiment, the first strand cDNA is synthesized from an RNA population using an oligonucleotide primer complex, i.e., an oligonucleotide primer linked to a promoter region. In this embodiment, the oligonucleotide primer will be substantially complementary to a section of the RNA, typically at the 3′ terminus. The promoter region is located upstream of the oligonucleotide primer at its 5′ terminus in an orientation permitting transcription with respect to the RNA population utilized. This will usually, but not always, mean that the promoter DNA sequence operably linked to the oligonucleotide primer is the complement to the functional promoter sequence. When the second strand cDNA is synthesized, as described below, the promoter sequence will be in correct orientation in that strand to initiate RNA synthesis using the second strand cDNA as a template. In one embodiment, the promoter region is derived from a prokaryote, such as SP6, T3 and T7 phages.

The oligonucleotide primer is preferably, but is not limited to, a single stranded oligodeoxynucleotide. The oligonucleotide primer must be sufficiently long to act as a template for the synthesis of extension products in the presence of the replicating agent. The exact lengths of the oligonucleotide primers and the quantities used will depend on many factors, including the temperature and degree of homology. Generally, the oligonucleotide primers will contain about 6 to about 30 nucleotides. In one embodiment, the oligonucleotide primers will contain about 10 to about 30 nucleotides. In yet another embodiment, the oligonucleotide primers will contain about 18 to about 25 nucleotides.

Once the oligonucleotide primer complex hybridizes to the RNA, a first strand CDNA is synthesized. In one embodiment, this first strand cDNA is produced utilizing a reverse transcription reaction, wherein cDNA is made from RNA following standard techniques. In a reverse transcription reaction, a reverse transcriptase adds deoxyribonucleotides to the 3′ terminus of the hybridized oligonucleotide primer.

The second strand cDNA, creating double-stranded cDNA, may be synthesized by any means known in the art. In one embodiment, RNase H and E. coli DNA polymerase are utilized. RNase assists breaking the RNA/first strand cDNA hybrid. DNA polymerase synthesizes a complementary DNA strand from the template first strand cDNA. The second strand cDNA is generated as dexoyribonucleotides are added to the 3′ terminus of the growing strand. As the growing strand reaches the 5′ terminus of the first strand DNA, the complementary promoter region of the first strand will be copied into the double stranded promoter sequence in the desired orientation.

In one embodiment, the species of interest within the RNA population is polyadenylated RNA. In this embodiment, the oligonucleotide primer complex may contain an oligonucleotide poly d(T) primer in conjunction with the promoter sequence. The oligonucleotide poly d(T) primer may comprise anywhere from about 10 to 30 thymidine residues. In another embodiment, the oligonucleotide poly d(T) primer comprises about 18 to 25 thymidine residues. The oligonucleotide poly d(T) primer will bind with the polyadenylate tail present on the 3′ terminus of each polyadenylated RNA molecule.

Generation of a cRNA Population from a cDNA Population

The cRNA population may be obtained by in vitro transcription from the cDNA population. The second cDNA strand is transcribed into cRNA, which is the complement of the initial RNA population. Amplification occurs because the polymerase repeatedly recycles on the same template (i.e., reinitiates transcription from the promoter region). Recycling of the polymerase on the same template avoids propagation of errors.

The RNA polymerase used for the transcription must be capable of operably binding to the particular promoter region employed in the oligonucleotide primer complex. Any polymerase/promoter combination can be used, provided the polymerase has specificity for that promoter in vitro sufficient to initiate transcription.

In one embodiment, T7 RNA polymerase is utilized in the presence of the appropriate NTPs for the transcription reaction.

Generation of a Sense cDNA Population from an Antisense cRNA Population

The cDNA population may be generated from the antisense cRNA population by any means known in the art.

In one embodiment, the first strand cDNA is synthesized from an antisense cRNA population using an oligonucleotide primer complex, i.e., an oligonucleotide primer linked to a promoter region. In this embodiment, the oligonucleotide primer will be substantially complementary to a section of the antisense cRNA, typically at the 3′ terminus. The promoter region is located upstream of the oligonucleotide primer at its 5′ terminus in an orientation permitting transcription with respect to the antisense cRNA population utilized. This will usually, but not always, mean that the promoter DNA sequence operably linked to the oligonucleotide primer is the complement to the functional promoter sequence. When the second strand cDNA is synthesized, as described below, the promoter sequence will be in correct orientation in that strand to initiate RNA synthesis using the second strand cDNA as a template. In one embodiment, the promoter region is derived from a prokaryote, such as SP6, T3 and T7 phages.

The oligonucleotide primer is preferably, but is not limited to, a single stranded oligodeoxynucleotide. The oligonucleotide primer must be sufficiently long to act as a template for the synthesis of extension products in the presence of the replicating agent. The exact lengths of the oligonucleotide primers and the quantities used will depend on many factors, including the temperature and degree of homology. Generally, the oligonucleotide primers will contain about 6 to about 30 nucleotides. In one embodiment, the oligonucleotide primers will contain about 10 to about 30 nucleotides. In yet another embodiment, the oligonucleotide primers will contain about 18 to about 25 nucleotides.

Once the oligonucleotide primer complex hybridizes to the antisense cRNA, a first strand cDNA is synthesized. In one embodiment, this first strand cDNA is produced utilizing a reverse transcription reaction, wherein cDNA is made from antisense cRNA following standard techniques. In a reverse transcription reaction, a reverse transcriptase adds deoxyribonucleotides to the 3′ terminus of the hybridized oligonucleotide primer.

The second strand cDNA, creating double-stranded cDNA, may be synthesized by any means known in the art. In one embodiment, RNase H and E. coli DNA polymerase are utilized. RNase assists breaking the antisense cRNA/first strand cDNA hybrid. DNA polymerase synthesizes a complementary DNA strand from the template first strand cDNA. The second strand cDNA is generated as deoxyribonucleotides are added to the 3′ terminus of the growing strand. As the growing strand reaches the 5′ terminus of the first strand DNA, the complementary promoter region of the first strand will be copied into the double stranded promoter sequence in the desired orientation.

In one embodiment, the species of interest within the antisense cRNA population is a polyadenylated RNA. In this embodiment, the oligonucleotide primer complex may contain an oligonucleotide poly d(T) primer in conjunction with the promoter sequence. The oligonucleotide poly d(T) primer may comprise anywhere from about 10 to 30 thymidine residues. In another embodiment, the oligonucleotide poly d(T) primer comprises about 18 to 25 thymidine residues. The oligonucleotide poly d(T) primer will bind with the polyadenylate tail present on the 3′ terminus of each polyadenylated RNA molecule.

Generation of an Antisense cDNA Population from a Sense cDNA Population

The antisense cDNA population may be generated from the sense cDNA population by any means known in the art.

In one embodiment, the synthesis of antisense cDNA from sense cDNA utilizes a molecule with polymerase activity that will synthesize primer extension products, including enzymes. Suitable enzymes for this purpose include, but are not limited to, DNA polymerase I, the Klenow fragment, such as from E. coli DNA polymerase I, T4 DNA polymerase, other available DNA polymerases, reverse transcriptase, and other enzymes, including heat-stable enzymes. The suitable molecule will facilitate combination of the nucleotides in the proper manner to form the primer extension products that are complementary to each nucleic acid strand. Generally, the synthesis will be initiated at the 3′ end of each oligonucleotide primer and proceed in the 5′ direction along the template strand, until synthesis terminates. As a result, molecules of different lengths are produced.

The oligonucleotide primer is preferably, but is not limited to, a single stranded oligonucleotide. The oligonucleotide primer must be sufficiently long to act as a template for the synthesis of extension products in the presence of the replicating agent. The exact lengths of the oligonucleotide primers and the quantities used will depend on many factors, including the temperature and degree of homology. Generally, the oligonucleotide primer will contain about 6 to about 30 nucleotides. In one embodiment, the oligonucleotide primer will comprise a random oligonucleotide. In another embodiment, the oligonucleotide primer will comprise a random hexamer.

In one embodiment of the present invention, the multiple copies of the antisense cDNA are labeled during the antisense cDNA synthesis. Alternatively, labeling of the antisense cDNA may occur following the synthesis via the attachment of a detectable label in the presence of terminal transferase.

Labels suitable for use in the present invention include any composition detectable by spectroscopic, photochemical, biochemical, immunochemical, electrical, optical, or chemical means. The label may be any suitable labeling substance, including but not limited to, a radioisotope, an enzyme, an enzyme cofactor, an enzyme substrate, a dye, a hapten, a chemiluminescent molecule, a fluorescent molecule, a phosphorescent molecule, an electrochemiluminescent molecule, a chromophore, a base sequence region that is unable to stably hybridize to the target nucleic acid under the stated conditions, and mixtures of these. Useful labels in the present invention include, but not limited to, biotin for staining with labeled streptavidin conjugate, magnetic beads, such as Dynabeads™, fluorescent dyes, such as fluoroscein, Texas red, rhodamine, and green fluorescent protein, radiolabels, such as ³H, ¹²⁵I, ³⁵S, ¹⁴C, and ³²P, enzymes, such as horse radish peroxidase and alkaline phosphatase, and calorimetric labels, such as colloidal gold and colored glass or plastic beads. In one embodiment, the label is biotin or a fluorescent label.

The labels may be detected using a variety of means known by a person of ordinary skill in the art. Radiolabels may be detected using photographic film or scintillation counters. Fluorescent markers may be detected using a photodetector to detect emitted light. Enzymatic labels may be detected by providing the enzyme with a substrate and detecting the reaction product produced by the action of the enzyme on the substrate. Calorimetric labels may be detected by simply visualizing the colored label.

Analysis Using a Nucleotide Assay

The antisense cDNA population may be utilized to analyze RNA levels in a tissue. Systems in which the labeled antisense cDNA population would be useful include a nucleotide array, a membrane blot, a microwell, a sample tube, a bead, a fiber, a TaqMan analysis, and an in situ hybridization. The nucleotide assay may also comprise nucleic acid probes in solution.

In one embodiment, the nucleotide assay comprises a nucleotide array. In this embodiment, the nucleotide array comprises nucleic acid probes bound to a solid support in known locations. Labeled antisense cDNA will hybridize to the probes if the labeled antisense cDNA contains a sequence complementary to a probe bound to the solid support. The nucleotide array may be prepared and utilized in any manner known in the art. Particular examples of methods for preparing a nucleotide array and conditions to be used when utilizing a nucleotide array are disclosed in U.S. Patent Application No. 2004/0259124 and U.S. Pat. Nos. 5,837,832; 5,889,165; 6,147,205; 6,262,216; 6,306,643 B1; 6,309,823 B1; 6,310,189 B1; 6,344,316 B1; and 6,410,229 B1, herein incorporated by reference.

The nucleotide array may be used to facilitate a comparative analysis of expression in different cells or tissues, different subpopulations of the same cells or tissues, different physiological states of the same cells or tissue, different developmental stages of the same cells or tissue, or different cell populations of the same tissue. In one embodiment, the amplification methods of the present invention can provide reproducible results (i.e., within statistically significant margins of error or degrees of confidence) sufficient to facilitate the measurement of quantitative as well as qualitative differences in the tested samples. The amplification methods of the present invention may also facilitate the identification of SNPs (i.e., point mutations that can serve, for example, as markers in the study of genetically inherited diseases) and other genotyping methods from limited sources. The mapping of SNPs can occur by any of various methods known in the art.

In another embodiment, the nucleotide assay comprises a TaqMan analysis. The TaqMan analysis may be performed in any manner known in the art. In one embodiment, the TaqMan analysis may be performed by annealing a fluorogenic probe containing a reporter dye and a quencher dye to the generated unlabeled antisense cDNA. During the extension phase of the PCR cycle, the 5′-exonuclease activity of Taq polymerase will cleave the hybridized fluorogenic probe, releasing the reporter dye from the quencher. As a result, an increase in detectable fluorescence will be observed. Other embodiments of the TaqMan analysis are described in U.S. Pat. No. 5,210,015 and D. E. Watson & B. Li, “TaqMan Applications in Genetic and Molecular Toxicology,” Intl. J. Tox., 24:139-145 (May-June 2005).

The TaqMan analysis may be used to facilitate a comparative analysis of expression in different cells or tissues, different subpopulations of the same cells or tissues, different physiological states of the same cells or tissue, different developmental stages of the same cells or tissue, or different cell populations of the same tissue. In one embodiment, the amplification methods of the present invention can provide reproducible results (i.e., within statistically significant margins of error or degrees of confidence) sufficient to facilitate the measurement of quantitative as well as qualitative differences in the tested samples. The amplification methods of the present invention may also facilitate the identification of SNPs (i.e., point mutations that can serve, for example, as markers in the study of genetically inherited diseases) and other genotyping methods from limited sources. The mapping of SNPs can occur by any of various methods known in the art.

Alternatively, the antisense cDNA population may be used for construction of complex cDNA libraries from extremely limited amounts of tissue, such as individual brain nuclei, tissue sections, and even single cells. The cDNA library may be constructed by any means known in the art.

The following examples are illustrative, but not limiting, of the methods of the present invention. Other suitable modifications and adaptations of the variety of conditions and parameters normally encountered in the field, and which are obvious to those skilled in the art, are within the spirit and scope of the invention.

All patents and publications cited herein are fully incorporated by reference herein in their entirety.

### EXAMPLES

### Example 1

### Total RNA Sample Preparation

Universal Reference RNA representing a pool of 11 different mouse cell lines was purchased from Stratagene (La Jolla, Calif., USA). Embryo, embryo (fibroblast), kidney, liver (hepatocyte), lung (alveolar macrophage), B-lymphocyte, T-lymphocyte, mammary gland, muscle myoblast, skin and testis cell lines are represented. Total RNA was further cleaned using the Pico Pure RNA Isolation kit (Arcturus, Mountain View, Calif.) with optional on column DNase treatment with the RNase-Free DNase Set (Qiagen, Valencia, Calif.).

A stock solution was used as the starting source for serial dilutions according to the Affymetrix Eukaryotic Sample Analysis Technical Manual, Rev 5. Strictly in terms of mass, every 3 μl of stock solution contained 5 μg mouse universal RNA, 15 pg of dap, 4 pg of thr, 2 pg of phe, and 1 pg of lys. In brief, B. subtilis transcripts lys, phe, thr, and dap from the Eukaryotic Poly-A Control Kit (Affymetrix, Santa Clara, Calif.) were spiked into 5 μg mouse universal total RNA at a complexity ratio of 1:100,000, 1:50,000, 1:25,000, and 1:6,666, respectively.

Mass to copy number conversions were determined assuming an average transcript length of 2000 base pairs, a molecular weight of 330 grams per mol per base pair, and 2% MRNA component of total RNA.

A replicate is defined as a separate serial dilution series from the stock solution, as well as a separate master mix for all enzymatic reactions.

The Mouse Assorted Total RNA kit (Ambion, Austin, Tex.) was used in QRT-PCR validation experiments. Liver, brain, heart, lung, spleen, ovary, kidney, and embryo derived from Swiss Webster mice are represented in the Mouse Assorted Total RNA Kit.

Total RNA samples were further cleaned using RNeasy kit (Qiagen).

Isolation and purification of total RNA for flow sorting experiments was conducted using the RNAqueous-Micro kit (Ambion) with substitution of DEPC-treated water for nucleic acid elution in the final step.

### Example 2

### Amplification and Labeling of total RNA for Affymetrix GeneChips

GeneChip One-Cycle Target Labeling and Control Reagents (Affymetrix) and the recommended protocols from the Affymetrix Eukaryotic Sample Analysis Technical Manual, Rev 5 were utilized on 4 replicates of 1 μg dilution of mouse universal stock solution. Similarly, 1 μg samples of liver, brain, heart, lung, spleen, ovary, kidney, and embryo were processed. This protocol, referred to as AFFX 1rd, involves a single round of in vitro transcription (“IVT”) resulting in a biotinylated cRNA target.

GeneChip Two-Cycle Target Labeling and Control Reagents (Affymetrix) and Affymetrix Eukaryotic Sample Analysis Technical Manual, Rev 5 were utilized on 2 replicates of 500 ng, 50 ng, 5 ng, 0.5 ng, and 50 pg dilutions of mouse universal stock solution using 20 ng/μl polyl (Sigma, St. Louis, Mo.) as a carrier nucleic acid. This protocol, referred to as AFFX 2rd, involves two rounds of in vitro transcription resulting in a biotinylated cRNA target.

RiboAmp HS RNA Amplification kit (Arcturus) and recommended protocols from laboratory manual version C were utilized on 2 replicates of 500 ng, 50 ng, 5 ng, 0.5 ng, and 50 pg dilutions of mouse universal stock solution using 20 ng/μl poly I (Sigma, St. Louis, Mo.) as a carrier nucleic acid. This protocol, referred to as ARC HS, involves two rounds of in vitro transcription resulting in a biotinylated cRNA target.

NuGEN Ovation Biotin-RNA Amplification and Labeling System, version 1.0 (NuGEN, San Carlos, Calif.) was utilized according to recommended protocols on 2 replicates of 500 ng, 50 ng, 5 ng, 0.5 ng, and 50 pg dilutions of mouse universal stock solution. This protocol, referred to as NGN, involves a single round, linear procedure utilizing a DNA/RNA chimeric primer and DNA polymerase to generate a biotinylated cDNA target.

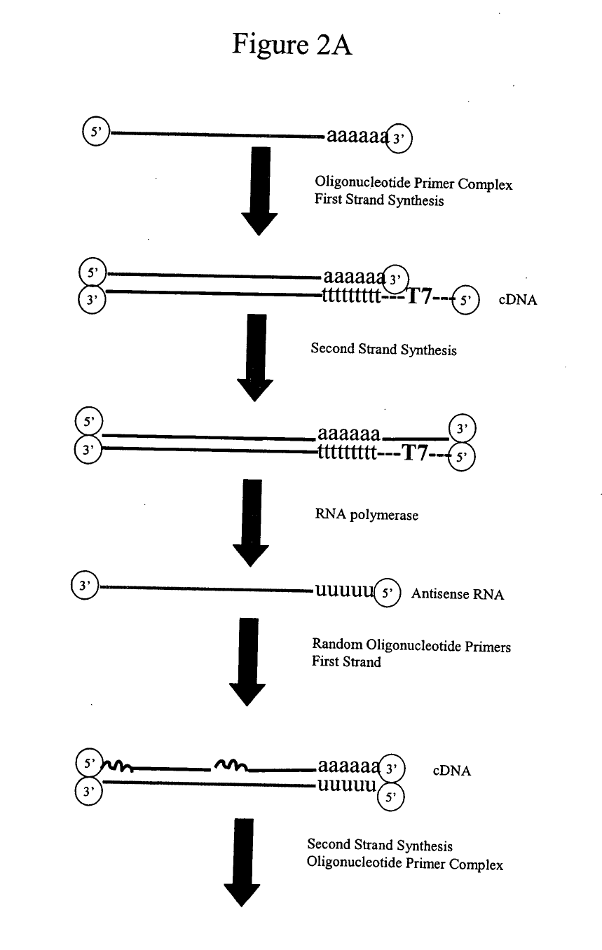

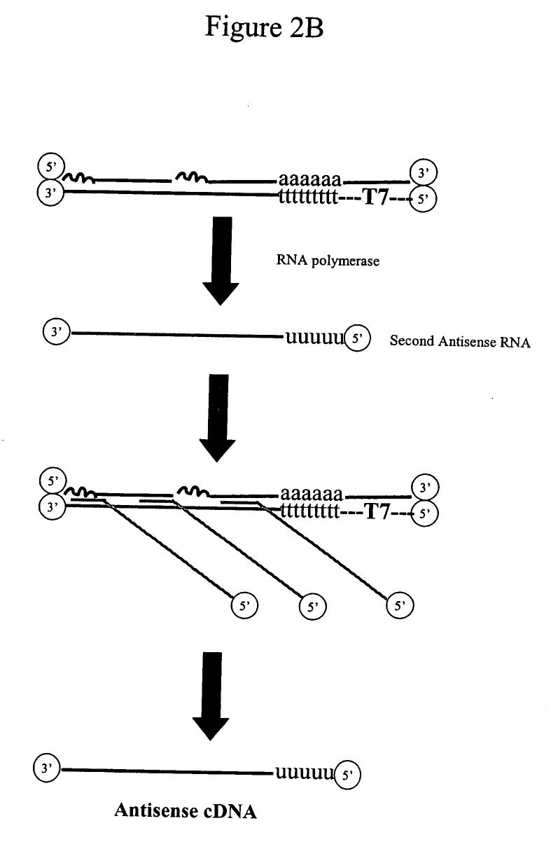

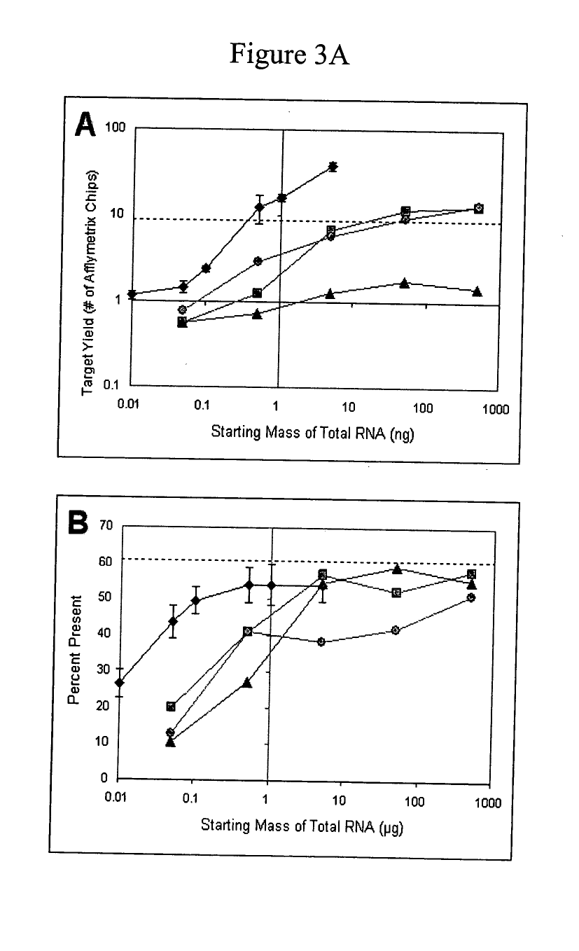

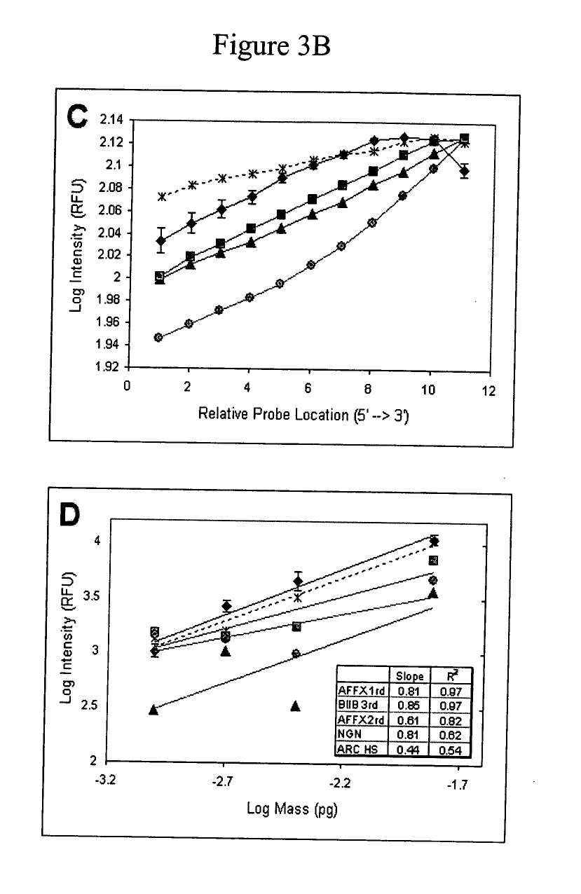

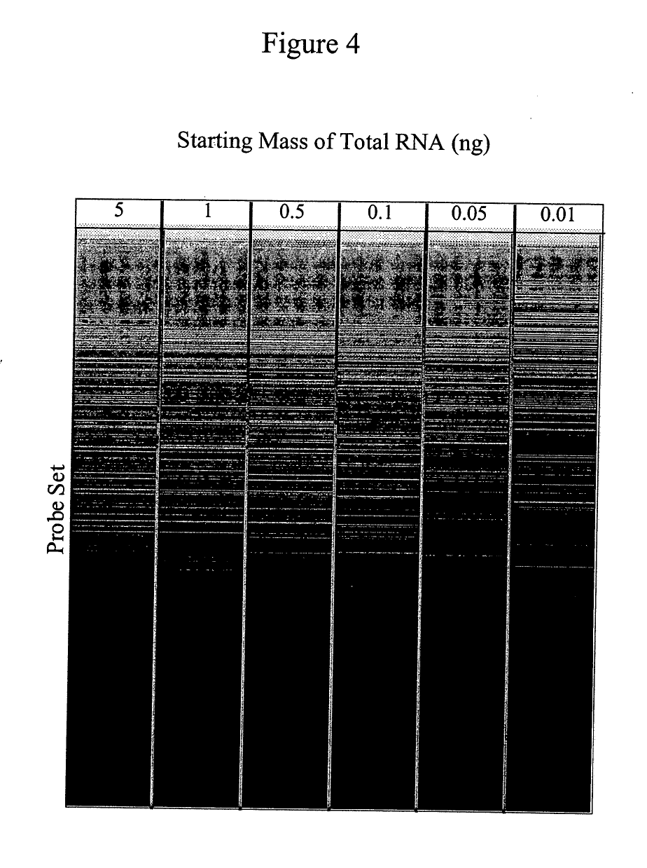

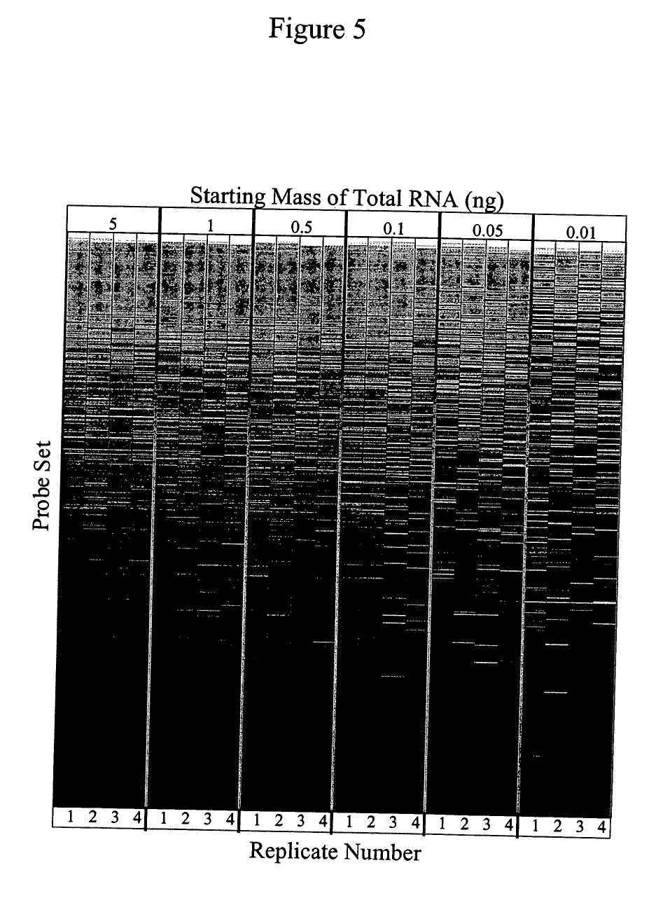

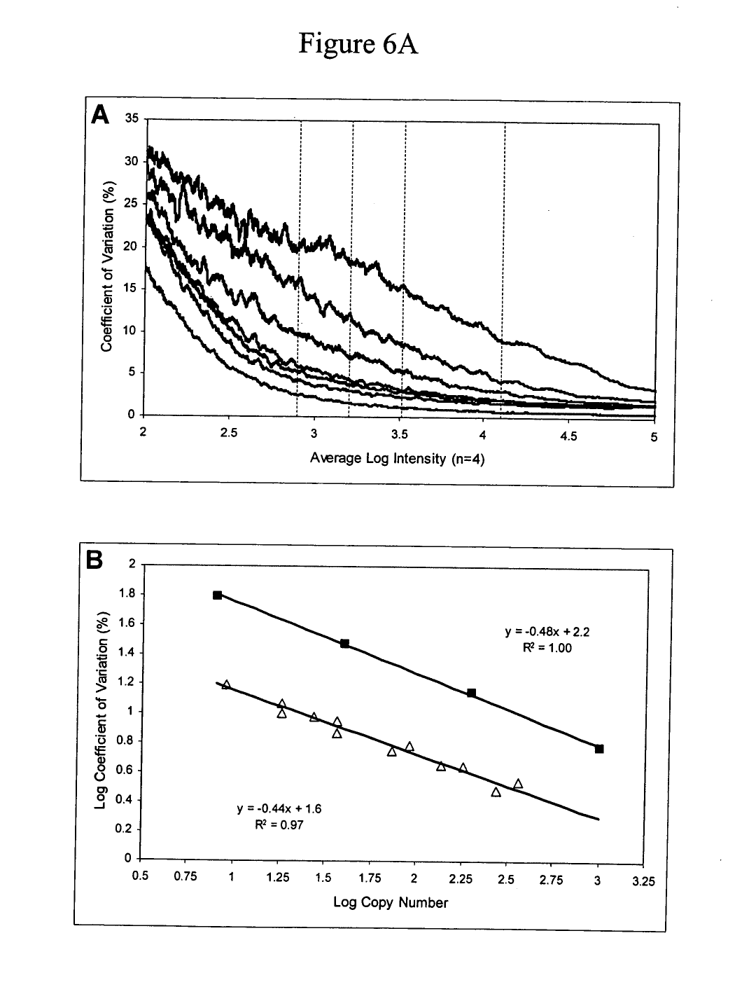

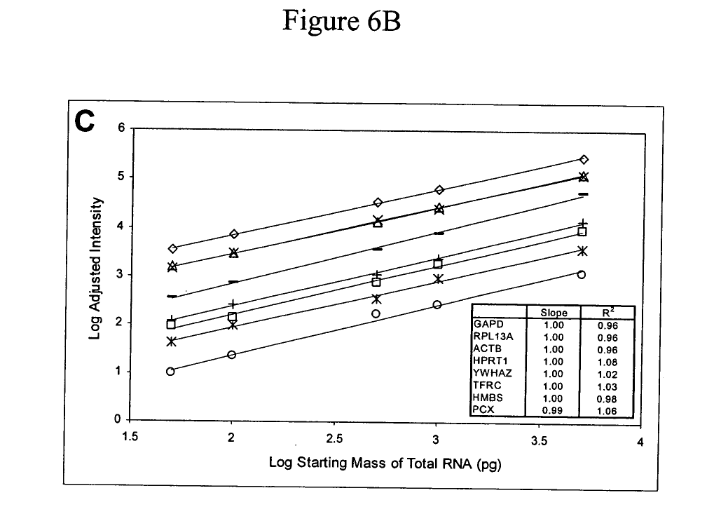

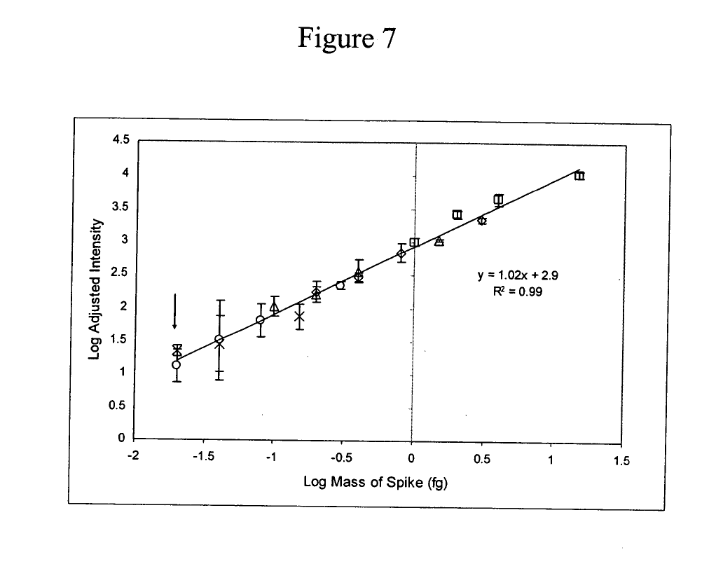

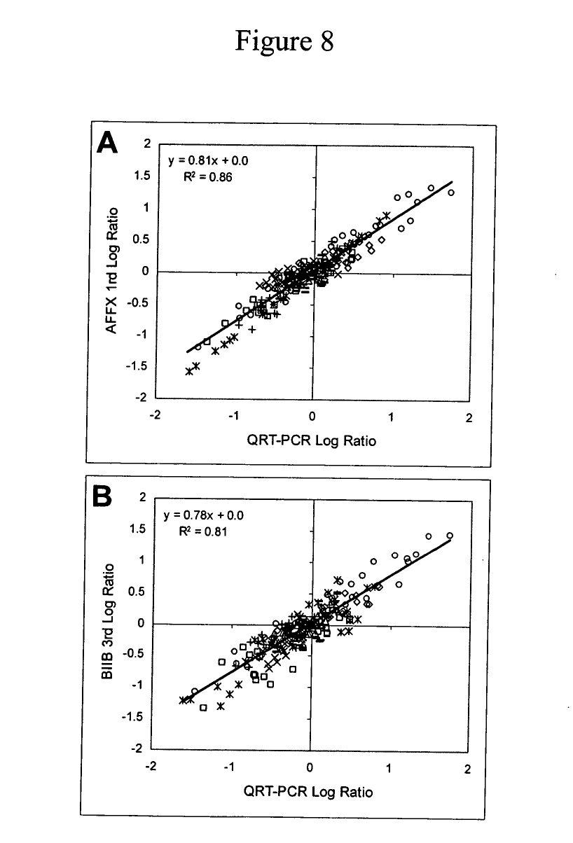

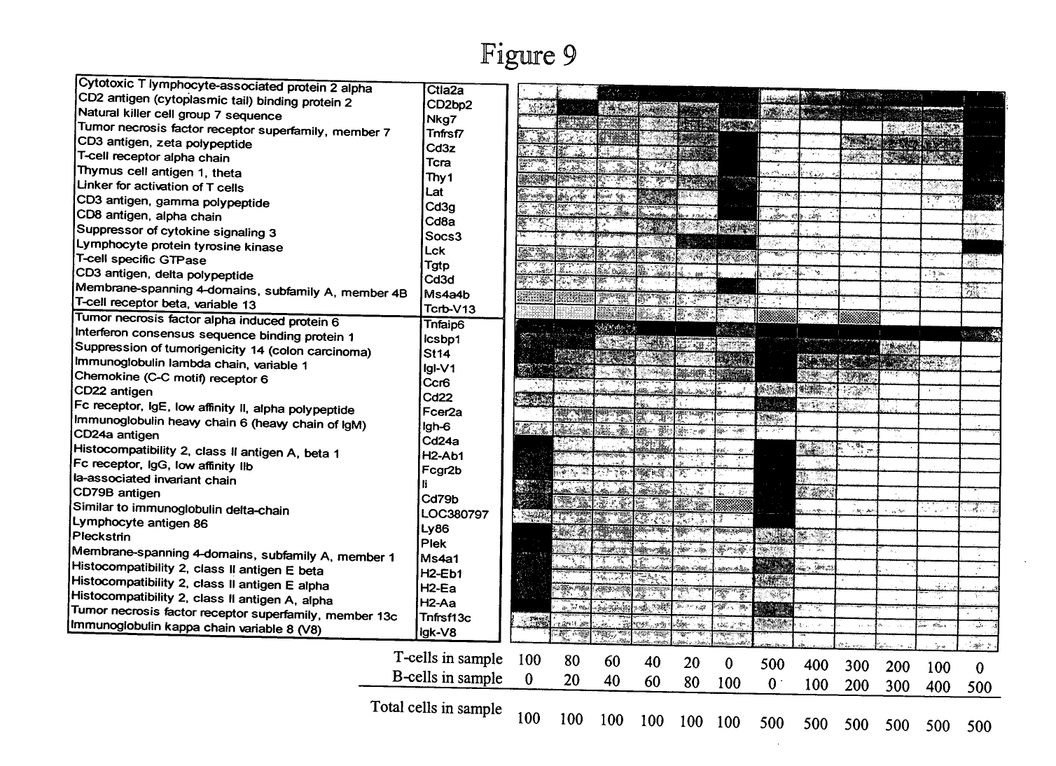

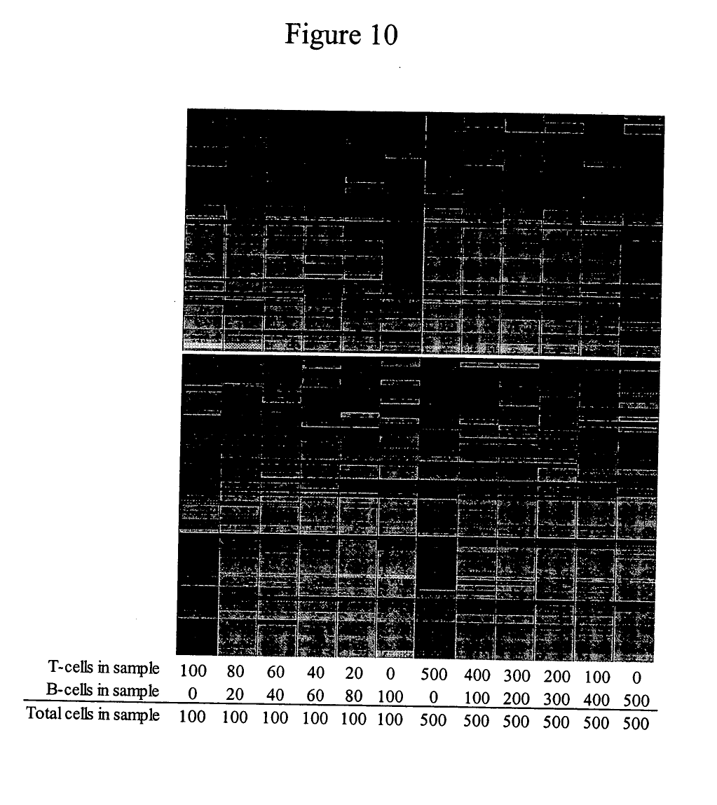

The amplification method of the present invention, as shown in FIG. 2, is comprised of two rounds of in vitro transcription, followed by a randomly primed first strand cDNA reaction. Klenow fragment of DNA Polymerase I is then used in a second strand cDNA synthesis to create a biotinylated cDNA probe. The amplification method of the present invention, as shown in FIG. 2, was utilized on 4 replicates at 5 ng, 1 ng, 0.5 ng, 100 pg, 50 pg, and 10 pg dilutions of mouse universal stock solution. Single samples using 500 pg of total RNA from liver, brain, heart, lung, spleen, ovary, kidney, and embryo was also processed using the method of the present invention. Similarly, the amplification method of the present invention was used on 12 samples containing the total RNA purified from 100 primary cells and 12 total RNA samples purified from 500 primary cells.

### Example 3

### Amplification and Labeling Protocol of the Present Invention

Unless otherwise noted, reagents from the GeneChip Two-Cycle cDNA Synthesis kit (Affymetrix) were utilized. Alternatively, enzymatic reagents (Invitrogen Corporation, Carlsbad, Calif.) may be substituted. All enzymatic reactions with the exception of the final Klenow labeling were carried out in a 96-well MJ Research thermocycler (Bio-Rad Laboratories, Hercules, Calif.).

IVT Round 1: The total RNA, suspended in 3 μl of DEPC-treated water, was supplemented with 2 μl of 5 μM T7-Oligo(dT) Primer, heated to 70° C. for 6 min, and cooled to 4° C. First strand master solution containing 2 μl of 5×1^{st} Strand Rxn Mix, 1 μl of 0.1 M DTT, 0.5 μl of 10 mM dNTP, 0.5 μl of RNase Inhibitor, and 1 μl of SuperScript II enzyme was added to the total RNA, then incubated for 1 hr at 42° C., followed by 10 min at 70° C., and finally cooled to 4° C. Second strand master mix containing 4.8 μl DEPC-treated water, 4.0 μl of 17.5 mM MgCl₂, 0.4 μl of 10 mM dNTP, 0.6 μl of E. coli DNA Polymerase I, and 0.2 μl of RNase H was added to the first strand reaction, incubated for 2 hr at 16° C. with the thermocycler heated lid disabled, heated to 75° C. for 10 min, and cooled to 4° C. MEGAscript T7 kit (Ambion) was used to assemble the IVT master mix containing 5 μl each of 10× Reaction Buffer, Enzyme Mix, and ATP, CTP, UTP, and GTP solutions. This solution was added directly to the product of the second strand reaction and incubated for 16 hr at 37° C.

IVT Round 2: IVT product was purified using cRNA Cleanup Spin Columns and protocols found in the Affymetrix Sample Cleanup Module. Purification was conducted according to Affymetrix Eukaryotic Sample Analysis Technical Manual, Rev 5, with the exception of elution, which used 20 μl of DEPC-treated water. When starting with 50 ng of total RNA, the concentration of cRNA sample was determined and a 300 ng aliquot removed for further processing. The volume of cRNA product was reduced to 4 μl by vacuum centrifugation, supplemented with 1 μl of 0.2 μg/μl Random Primers, heated to 70° C. for 6 min, and cooled to 4° C. First strand master solution was formulated and added to the purified cRNA as described for IVT Round 1. First strand reaction was incubated at 42° C. for 1 hr, followed by addition of 0.5 μl RNase H prior to incubation for 20 min at 37° C., and finally enzyme inactivation at 95° C. for 5 min. 2 μl of 5 μM of T7-Oligo(dT) Primer was added to the first strand reaction. The solution was denatured at 70° C. for 6 min and cooled to 4° C. Second strand master mix containing 2.5 μl DEPC-treated water, 4.0 μl of 17.5 mM MgCl₂, 0.4 μl of 10 mM dNTP, and 0.6 μl of E. coli DNA Polymerase I was added to the first strand reaction and incubated for 2 hr at 16° C. with the thermocycler heated lid disabled. Next, 0.5 μl of T4 DNA Polymerase was added. Incubation proceeded at 16° C. with thermocycler heated lid disabled, followed by inactivation at 75° C. for 10 min before cooling to 4° C. Second round IVT master mix was prepared as above, added directly to the product of the second strand reaction, and incubated for 16 hr at 37° C.

Klenow Labeling: The IVT product was cleaned as described above, with the exception of elution, which was carried out using 50 μl of DEPC-treated water. The concentration of cRNA was determined. A 2 μg aliquot of cRNA was removed, vacuum centrifuged to 8 μl, and used as the template for cDNA production via the SuperScript III First-Strand Synthesis System for RT-PCR (Invitrogen). The cRNA was supplemented with 1 μl of dNTP mix and 1 μl of random oligonucleotide hexamers, denatured at 65° C. for 5 min, and cooled at 4° C. First strand master solution containing 2 μl of 10× RT Buffer, 4 μl of 25 mM MgCl₂, 2 μl of 0.1 M DTT, 1 μl of RNaseOUT, and 1 μl of SuperScript III enzyme was added to cRNA. The mixture was then incubated at 25° C. for 10 min, 42° C. for 50 min, 85° C. for 5 min, and finally cooled to 4° C. The cRNA template was degraded by adding 1 μl of RNase H and incubating at 37° C. for 20 min. The second strand cDNA synthesis was completed via the BioPrime DNA Labeling System (Invitrogen). The entire first strand cDNA reaction was combined with 100 μl of the 2.5× Random Primers Solution and 29 μl of water. The sample was then heated to 95° C. for 5 min followed by 5 min of chilling at 4° C. A master mix containing 25 μl of 10× dNTP Mixture (1 mM biotin-14-dCTP, 2 mM dATP, 2 mM dGTP, and 2 mM dTTP), 70 μl of water, and 5 μl of Klenow Fragment was added to the first strand cDNA solution and incubated at 37° C. for 2 hr. Finally, 25 μl of Stop Buffer was added to each sample and frozen at −20° C. overnight.

Second Strand cDNA Purification: The second strand cDNA reaction was transferred to a 15 ml conical tube containing 2.5 ml of Buffer PN from the QIAquick Nucleotide Removal Kit (Qiagen). Successive 750 μl aliquots of cDNA/PN solution were added to a QIAquick column using a QIAvac 24 vacuum manifold (Qiagen). Column-bound cDNA was washed with 1 ml of Buffer PE, dried completely using centrifugation at 12,000 RCF for 5 min, and eluted in 70 μl of H₂O.

### Example 4

### Target Hybridization to Affymetrix GeneChips

The cRNA targets generated from the AFFX 1rd, AFFX 2rd, and ARC HS protocols, were fragmented according to the Affymetrix Eukaryotic Sample Analysis Technical Manual, Rev 5 using volumes scaled to accommodate 11 μg of input target RNA. The cDNA generated from the NGN procedure was fragmented and biotin labeled according to NuGEN recommendations to yield 2.2 μg cDNA target. The product of the entire amplification reaction of Example 4 was used as the cDNA target.

All of the samples were hybridized to the GeneChip according to the Affymetrix Eukaryotic Sample Analysis Technical Manual, Rev 5. In brief, the cDNA or the cRNA target was suspended in a final volume of 220 μl of 1× hybridization buffer supplemented with 0.5 mg/mL aceylated BSA, 0.1 mg/mL herring sperm DNA, control oligo B2, eukaryotic control transcripts, and 10% DMSO. 200 μl of each sample was hybridized overnight at 45° C. to a Mouse Genome 430A GeneChip. The samples were washed and stained on a GeneChip Fluidics Station 450 using the EukGE-WS2v4 script and visualized using a GeneChip Scanner 3000. For all of the samples, the statistical algorithm of Microarray Suite 5.0 (MAS 5.0) was used to calculate probe set expression intensities with a scaling factor of 2500 and served as the starting point for further analysis.

### Example 5

### QRT-PCR Experiments

Oligonucleotide primers (Biosearch Technologies, Novato, Calif.) and Taqman MGB (Applied Biosystems, Foster City, Calif.) probes were designed using Primer Express version 2.0.0 (Applied Biosystems) from the Affymetrix consensus sequence for the following housekeeper probe sets: 1436722_a_at (beta actin, ACTB), 1418625_s_at (glyceraldehyde-3-phosphate dehydrogenase, GAPD), 1426475_at (hydroxymethylbilane synthase, HMBS), 1448736_a_at (hypoxanthine guanine phosphoribosyl transferase 1, HPRT1), 1416383_a_at (pyruvate carboxylase, PCX), 1455485_x_at (ribosomal protein L13a, RPL13A), 1452661_at (transferrin receptor, TFRC), and 1448218_s_at (tyrosine 3-monooxygenase/tryptophan 5-monooxygenase activation protein, zeta, YWHAZ). Taqman MGB probes contain a 5′ covalently linked fluorescent reporter dye (FAM) and a minor groove binder non-fluorescent quencher (MGBNF) covalently linked to the 3′ end. Oligonucleotide standard template (Biosearch Technologies) design included 10 bp of gene specific sequence at the 5′ and 3′ ends of the amplicon.

The total RNA samples were concentration normalized by measuring absorbance at 260 nm. The residual DNA was removed from 5 μg of total RNA using 5 units DNase I amplification grade (Invitrogen) at 20° C. for 15 minutes. An aliquot of the treated sample was used as a control in the subsequent QRT-PCR assays to ensure the absence of DNA contamination. The remaining DNase treated RNA was used in a cDNA synthesis reaction using a high capacity cDNA archive kit (Applied Biosystems).

The oligonucleotide templates were pooled and then serially diluted 1 to 10 eight times in 25 ng/μl yeast RNA (Ambion) to include a final range 500 fM to 5 zM. Quadruplicate PCR reactions for samples and standards were mixed in a 96 well plate, transferred to a 384 well optical plate (Applied Biosystems), and cycled in a 7900HT (Applied Biosystems) thermalcycler under the following conditions: 50° C. for 2 minutes (uracil N-deglycosylase digest), 95° C. for 10 minutes (activation of Taq thermostable polymerase), and 40 cycles of 95° C. for 15 seconds and 60° C. for 60 seconds. Relative transcript quantities for each sample was determined by comparison to oligonucleotide standard curve using Sequence Detection Software (Applied Biosystems).

### Example 6

### Intra-Protocol and Inter-Protocol Precision

The top value in each cell of the table below is the number of probe sets designated as present by both methods. The bottom number represents the average R² value of all pair-wise intensity comparisons for the present probe sets. The probe set intensity was determined using MAS 5.0 algorithm scaling to 2500. For intra-protocol data (diagonal, bold) a probe set was used in the analysis if 50% of the replicates reported it as present. For inter-protocol data a probe set was included in the analysis if 50% of each protocol's replicates reported the called as present.

| | | Inventive | | | |
|---|---|---|---|---|---|
| | AFFX 1rd | Method | AFFX 2rd | NGN | ARC HS |
| | (n = 4) | (n = 4) | (n = 2) | (n = 2) | (n = 2) |
| AFFX 1rd | 13426 | 10914 | 13042 | 12135 | 9811 |
| (n = 4) | 0.97 | 0.56 | 0.82 | 0.45 | 0.58 |
| Inventive | 10914 | 11645 | 11830 | 11665 | 9395 |
| Method | 0.56 | 0.92 | 0.66 | 0.41 | 0.50 |
| (n = 4) | | | | | |
| AFFX 2rd | 13042 | 11830 | 13742 | 11594 | 9857 |
| (n = 2) | 0.82 | 0.66 | 0.95 | 0.52 | 0.68 |
| NGN | 12135 | 11665 | 11594 | 13550 | 8704 |
| (n = 2) | 0.45 | 0.41 | 0.52 | 0.86 | 0.36 |
| ARC HS | 9811 | 9395 | 9857 | 8704 | 10246 |
| (n = 2) | 0.58 | 0.50 | 0.68 | 0.36 | 0.80 |

1. A method for amplifying a nucleic acid population comprising
(a) generating a DNA population from said nucleic acid population;
(b) generating an antisense cRNA population from said DNA population;
(c) generating a sense cDNA population from said antisense cRNA population; and
(d) generating an antisense cDNA population from said sense cDNA population.

2. The method of claim 1, wherein a label is incorporated into the antisense cDNA population generated in step (d).

3. The method of claim 2, wherein the label is selected from a group consisting of biotin, fluorescent dye, and a radioactive label.

4. (canceled)

5. The method of claim 1, wherein the nucleic acid population comprises a plurality of different RNAs.

6. The method of claim 5, wherein the plurality of different RNAs comprises a plurality of different polyadenylated RNAs.

7. (canceled)

8. The method of claim 5, wherein the DNA population is generated in step (a) by (i) contacting the RNAs with a first oligonucleotide primer complex comprising an oligonucleotide primer and an RNA polymerase promoter; (ii) extending the oligonucleotide primer in a reaction mixture comprising reverse transcriptase to yield RNA:cDNA duplexes; and (iii) synthesizing second strand cDNA by incubating the RNA:cDNA duplexes with a reaction mixture comprising DNA polymerase.

9. (canceled)

10. (canceled)

11. The method of claim 1, wherein the sense cDNA population is generated in step (c) by (i) contacting the antisense cRNA population with random oligonucleotide primers and (ii) extending the random oligonucleotide primers in a reaction mixture comprising reverse transcriptase.

12. The method of claim 11, wherein the random oligonucleotide primers comprise random oligonucleotide hexamers.

13. The method of claim 1, wherein the antisense cDNA population is generated in step (d) by contacting the sense cDNA generated in step (c) with a reaction mixture comprising random oligonucleotide primers and a molecule with polymerase activity.

14. The method of claim 13, wherein the molecule is selected from a group consisting of DNA polymerase I and Klenow fragment of DNA polymerase I.

15. The method of claim 14, wherein the molecule is the Klenow fragment of DNA polymerase I.

16. (canceled)

17. A method for amplifying an RNA population comprising
(a) generating a cDNA population from said RNA population;
(b) generating an antisense cRNA population from said cDNA population;
(c) generating a sense cDNA population from said antisense RNA population;
(d) generating a second antisense cRNA population from said sense cDNA population;
(e) generating a second sense cDNA population from the second antisense cRNA population; and
(f) generating an antisense cDNA population from the second sense cDNA population.

18. The method of claim 17, wherein the RNA population comprises polyadenylated RNA.

19. (canceled)

20. The method of claim 17, wherein a label is incorporated into the antisense cDNA population generated in step (f).

21. The method of claim 20, wherein the label is selected from a group consisting of biotin, fluorescent dye, and a radioactive label.

22. (canceled)

23. The method of claim 17, wherein the cDNA population is generated in step (a) by (i) contacting the RNA population with a first oligonucleotide primer complex comprising an oligonucleotide primer and an RNA polymerase promoter; (ii) extending the oligonucleotide primer in a reaction mixture comprising reverse transcriptase to yield RNA:cDNA duplexes; and (iii) synthesizing second strand cDNA by incubating the RNA:cDNA duplexes with a reaction mixture comprising DNA polymerase.

24. (canceled)

25. (canceled)

26. The method of claim 17, wherein the sense cDNA population is generated in step (c) by (i) contacting the antisense cRNA population with random oligonucleotide primers and (ii) extending the random oligonucleotide primers in a reaction mixture comprising reverse transcriptase.

27. (canceled)

28. The method of claim 17, wherein the second sense cDNA population is generated in step (e) by (i) contacting the second antisense RNA population with random oligonucleotide primers and (ii) extending the random oligonucleotide primers in a reaction mixture comprising reverse transcriptase.

29. The method of claim 28, wherein the random oligonucleotide primers comprise random oligonucleotide hexamers.

30. The method of claim 17, wherein the antisense cDNA population is generated in step (f) by contacting the sense cDNA generated in step (e) with a reaction mixture comprising random oligonucleotide primers and a molecule with polymerase activity.

31. The method of claim 30, wherein the molecule is selected from a group consisting of DNA polymerase I and Klenow fragment of DNA polymerase I.

32. The method of claim 31, wherein the molecule is the Klenow fragment of DNA polymerase I.

33. (canceled)

34. A method for amplifying a total RNA sample comprising:
(a) contacting an RNA population comprising a plurality of different RNAs with a first oligonucleotide primer complex comprising an oligonucleotide primer and an RNA polymerase promoter;
(b) extending the oligonucleotide primer in a reaction mixture comprising reverse transcriptase to yield RNA:cDNA duplexes;
(c) synthesizing second strand cDNA by incubating the RNA:cDNA with a reaction mixture comprising DNA polymerase to yield cDNA;
(d) contacting the CDNA with random oligonucleotide primers; and
(e) generating antisense cDNA from the cDNA by extending the random oligonucleotide primers in a reaction mixture comprising a molecule with polymerase activity.

35. The method of claim 34, wherein between steps (c) and (d), the method further comprises:
(i) producing an antisense cRNA by incubating the cDNA in a reaction mixture comprising an RNA polymerase;
(ii) contacting the antisense cRNA with a reaction mixture comprising random oligonucleotide primers;
(iii) generating RNA:cDNA duplexes from the antisense cRNA by extending the random oligonucleotide primers in a reaction mixture comprising a reverse transcriptase;
(iv) contacting the cDNA with a second oligonucleotide primer complex comprising an oligonucleotide primer and an RNA polymerase promoter and extending the oligonucleotide primer to generate a second cDNA;
(v) producing a second antisense cRNA by an in vitro transcription reaction;
(vi) contacting the second antisense cRNA with random oligonucleotide primers; and
(vii) generating RNA:cDNA duplexes from the second antisense cRNA by extending the random oligonucleotide primers in a reaction mixture comprising a reverse transcriptase.

36. The method of claim 34, wherein the different RNAs comprise different polyadenylated RNAs.

37-39. (canceled)

40. The method of claim 34, wherein the molecule with polymerase activity utilized in step (c) is selected from a group consisting of DNA polymerase I or the Klenow fragment of DNA polymerase I.

41. The method of claim 40, wherein the molecule with polymerase activity is the Klenow fragment of DNA polymerase I.

42. (canceled)

43. The method of claim 34, wherein the reaction mixture utilized in step (e) also comprises a label.

44. The method of claim 43, wherein the label is selected from a group consisting of biotin, a fluorescent label, and a radioactive label.

45. (canceled)

46. The method of claim 35, wherein the random oligonucleotide primers of steps (vi) and (vii) comprise random oligonucleotide hexamers.

47-62. (canceled)
